(19) United States
(12) Patent Application Publication
(10) Pub. No.: US 2008/0131390 A1
(43) Pub. Date: Jun. 5, 2008

(54) Hair Treatment Product Containing Anionic Surfactants and Cationic Polymers

(76) Inventor: Frank-Peter Lang, Hattersheim (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/667,436

(22) PCT Filed: Nov. 5, 2005
(86) PCT No.: PCT/EP05/11853

§ 371 Date: Jan. 22, 2008

(30) Foreign Application Priority Data
Nov. 9, 2004 (DE) 10 2004 053 968.5

(51) Int. Cl. A61K 8/72; A61Q 5/02

(52) U.S. Cl. 424/70.11

(57) ABSTRACT

The invention relates to a hair treatment product, especially shampoo, containing al) alkyl ether sulfate or a) alkyl sulfate or the mixture thereof, b) secondary alkane sulfonate, c1) betaine or c2) ether carboxylate or the mixture thereof, d) at least one non-ionic surfactant, and e) a cationic polymer from the group of homopolymers or copolymers of diallyl-dimethyl-ammonium chloride or the reaction products of cyanamides with aldehydes and ammonium salts, or cyanamides with aldehydes and monoamines, or monoamines and/or polyamines with epichlorohydrine, or polyamines with cyanamides and amidosulphuric acid.

The invention relates to cleansing hair treatment products, for example hair shampoos, comprising both anionic surfactants and cationic polymers.

Frequent bleaching, perming and coloring, but also frequent washing of the hair with degreasing surfactants, results in damage to the hair structure. The hair becomes brittle and loses its shine. In addition, the hair becomes electrostatically charged during combing, and the roughened, degreased hair surface causes matting and knotting of the hair. This makes combing more difficult. Despite this, many consumers wish to wash their hair daily on account of an increased need for hygiene.

To care for the hair, after hair washing, after-treatment products are often applied in a second step. As so-called hair cures, in the form of haircare rinses, aerosol foams or else in emulsion form as so-called cream rinses, these are rinsed out of the hair with water after a contact time of a few minutes. Leave-on products, e.g. in the form of conditioner sprays, hair fluids or styling products, for shaping and care, remain on the hair.

As active ingredients for improving the hair structure, such products comprise, inter alia, cationic polymers or cationic surfactants, i.e. quaternary ammonium compounds, such as cetyltrimethylammonium chloride on its own or in combination with various wax-like additives, such as, for example, hydrocarbons, fatty alcohols and fatty acids, which attach to the hair and improve its combability and shine.

On account of the consumer need for quicker and easier haircare, so-called two-in-one products have also been developed, through which hair washing and care take place at the same time. A series of care active ingredients is available for producing such conditioning shampoos.

These include oils and oil-like substances, such as, for example, liquid hydrocarbon compounds, fatty alcohols, monocarboxylic acid esters, polyalcohol esters, silicones, both soluble silicones, such as, for example, dimethicone copolyols, and insoluble silicones, for example polydimethyl-siloxanes, and cationic surface-active compositions and cationic polymers.

Hair shampoos comprising anionic surfactants and cationic polymers, however, cannot be formulated to be clear and stable on account of the reaction of these components to form electroneutral salts.

It is an object of the present invention to provide cleansing and simultaneously conditioning, care hair treatment products which comprise both anionic surfactants and cationic polymers and, despite the incompatibility of the components, are stable and form clear solutions.

Surprisingly, it has now been found that this aim can be achieved through a surfactant system in which the following are present: the anionic surfactants alkyl ether sulfate and/or alkyl sulfate, the anionic surfactant secondary alkanesulfonate, betaine and/or alkyl ether carboxylate, a nonionic surfactant.

The invention thus provides hair treatment products, preferably shampoos, comprising

a1) alkyl ether sulfate or

a2) alkyl sulfate or mixture thereof

b) secondary alkanesulfonate,

c1) betaine or

c2) ether carboxylate or mixture thereof,

d) one or more nonionic surfactants and

e) one or more cationic polymers from the group of homopolymers and copolymers of diallyidimethylammonium chloride, or

the reaction products of cyanamides with aldehydes and ammonium salts

or cyanamides with aldehydes and monoamines

or monoamines and/or polyamines with epichlorohydrin

or polyamines with cyanamides and amidosulfuric acid.

The invention further provides the use of a mixture of said mixture of components a1) or a2), b), c1) or c2), d) and e) as surfactant system in hair treatment products.

The individual components are described below:

a1) Alkyl Ether Sulfates

These are water-soluble salts or acids of the formula RO(A)ₘSO₃M in which R is an unsubstituted C₁₀-C₂₄-alkyl or hydroxyalkyl radical, preferably a C₁₂-C₂₀-alkyl or hydroxyalkyl radical, particularly preferably C₁₂-C₁₈-alkyl or hydroxyalkyl radical.

A is an ethoxy or propoxy unit, m is a number greater than 0, preferably between about 0.5 and about 6,particularly preferably between about 0.5 and about 3, and M is a hydrogen atom or a cation, such as, for example, sodium, potassium, lithium, calcium, magnesium, ammonium or a substituted ammonium cation. Specific examples of substituted ammonium cations are methyl-, dimethyl-, trimethylammonium and quaternary ammonium cations, such as tetramethylammonium and dimethylpiperidinium cations, and those which are derived from alkylamines, such as ethylamine, diethylamine, triethylamine or mixtures thereof. Examples which may be mentioned are C₁₂ to C₁₈-fatty alcohol ether sulfates, where the content of EO is 1, 2, 2.5, 3 or 4 mol per mole of the fatty alcohol ether sulfate, and in which M is sodium or potassium.

a2) Alkyl Sulfates

These are water-soluble salts or acids of the formula ROSO₃M, in which R is a C₁₀-C₂₄-hydrocarbon radical, preferably a C₁₀-C₂₀-alkyl or hydroxyalkyl radical, particularly preferably a C₁₂-C₁₈-alkyl or hydroxyalkyl radical.

M is hydrogen or a cation, e.g. an alkali metal cation (e.g. sodium, potassium, lithium) or ammonium or substituted ammonium, e.g. methyl-, dimethyl- and trimethylammonium cations, alkanolammonium, e.g. triethanolammonium and quaternary ammonium cations, such as tetramethylammonium and dimethylpiperidinium cations and quaternary ammonium cations, derived from alkylamines such as ethylamine, diethylamine, triethylamine and mixtures thereof.

Components a1 and a2 are used individually or in any combination with one another in concentrations of from 1 to 20% by weight, preferably 2 to 15% by weight, particularly preferably 3 to 12% by weight and very particularly preferably 4 to 10% by weight.

b) Secondary Alkanesulfonates.

In secondary alkanesulfonates, the alkyl group can either be saturated or unsaturated, branched or linear and optionally substituted by a hydroxyl group. The sulfo group can be at any position of the C chain, where the primary methyl groups at the start of the chain and the end of the chain have no sulfonate groups. The preferred secondary alkanesulfonates contain linear alkyl chains having about 9 to 25 carbon atoms, preferably about 10 to about 20 carbon atoms and particularly preferably about 13 to 17 carbon atoms. The cation is, for example, sodium, potassium, ammonium, mono-, di- or triethanolammonium, calcium or magnesium. It is also possible to use mixtures of different cations.

Very particular preference is given to secondary C₁₃₋₁₇-alkanesulfonate, Na salt, which is available, for example, under the trade name Hostapur® SAS (Clariant), Leuna-Alkansulfonat or Emulgator E30 (Leuna-Tenside GmbH) or Marlon® PS (Sasol).

Secondary alkanesulfonates are used additionally to components a1 and/or a2. The mixing ratio of sec. alkanesulfonate:(a1 and/or a2) is here generally

9.9:0.1 parts by weight,

preferably 9.8 to 5:0.2 to 5 parts by weight,

particularly preferably 4.9 to 2.5:5.1 to 7.5 parts by weight, and very particularly preferably 2.4 to 0.1:7.6 to 9.9 parts by weight.

c1) Betaines

Betaines can either be carbobetaines or sulfobetaines.

Preference is given to the carboxymethylammonium betaines, in particular C₈- to C₁₈-alkyldimethylcarboxymethylammonium betaines, C₈- to C₁₅-alkylamidopropyidimethylcarboxymethylammonium betaines and C₈- to C₁₈-alkyldipolyethoxycarboxymethylammonium betaines.

Further suitable betaines are, for example, the N-carboxyethylammonium betaines analogous to the compounds listed above, for whose synthesis chloropropionic acid and salts thereof are used instead of chloroacetic acid or salts thereof. Examples thereof are the C₁₂-C₁₈-alkylaminopropionates and C₁₂-C₁₈-alkyliminodipropionates as alkali metal and mono-, di- and trialkylammonium salts.

A preferred sulfobetaine is C₁₂-C₁₈-alkyldimethylsulfopropylbetaine.

c2) Ether Carboxylate

Ether carboxylates preferably have the general formula

R—(O—CH₂—CH₂)ₙ—O—CH₂—COOX

where R can be n-alkyl, isoalkyl, cycloalkyl, alkylaryl, arylalkyl and/or aryl.

Preferably, the radical R contains 8 to 18 carbon atoms and is derived from fatty alcohols, Ziegler alcohols, oxo alcohols, Guerbet alcohols and alkyl-phenols. Preference is given here to C₈- to C₁₈-alkyl ether carboxylates, and octyl-, nonyl-, and tributylphenol ether carboxylic acids.

Particularly preferred radicals R are C₈-alkyl, C_{12/14}-alkyl, iso-C₁₃-alkyl, C_{14/15}-oxoalkyl, C_{16/18}-alkyl, lauryl, oleyl, and/or tallow fatty alkyl. The index n=1 to 20, preferably 1 to 15, particularly preferably 1 to 12 and very particularly preferably 2 to 10.

X is hydrogen, sodium, potassium, ammonium and/or alkanolammonium, in particular triethanolammonium.

In the hair treatment products according to the invention, components c1 and c2 are used individually or in combination in a concentration of from 0.5 to 15% by weight,

d) Nonionic Surfactants.

Suitable nonionic surfactants are, in particular, the ethoxylates of long-chain, aliphatic, synthetic or native alcohols with a C₈- to C₂₂-alkyl radical. These can contain about 1 to about 25 mol of ethylene oxide.

The alkyl chain of the aliphatic alcohols can be linear or branched, primary or secondary, saturated or else unsaturated.

Preference is given to the condensation products of C₁₀- to C₁₈-alcohols with about 2 to about 18 mol of ethylene oxide per mole of alcohol. The alcohol ethoxylates can have a narrow homolog distribution of the ethylene oxide (“narrow range ethoxylates”) or a broad homolog distribution of the ethylene oxide (“broad range ethoxylates”).

Particular preference is given to the C₉-C₁₁ oxo alcohol with 6 to 10 mol of EO and the C_{12/14}-fatty alcohol with 5 to 9 mol of EO.

Very particular preference is given to the C₁₁-oxo alcohol-8EO-ethoxylate and the C_{12/14}-fatty alcohol-7EO-ethoxylate. The oxyethylated alcohols preferably have an HLB value of from 10 to 15, preferably 11 to 14.

The use concentration is generally 1 to 30% by weight, preferably 5 to 25% by weight and particularly preferably 10 to 20% by weight.

The quantitative ratio of anionic surfactant to nonionic surfactant is generally 1:4 to 4:1, preferably 1:2 to 2:1, very particularly preferably 0.8:1 to 1.5:1.

e) Cationic Polymers

Cationic polymers which are preferably used in the hair treatment products according to the invention are homopolymers and copolymers based on diallyidimethylammonium chloride (DADMAC).

Copolymers based on DADMAC comprise, as further components, other vinylic monomers, such as, for example, vinylimidazole, vinylpyrrolidone, vinyl alcohol, vinyl acetate, (meth)acrylic acid (ester), acrylamide, styrene, styrenesulfonic acid, acrylamidomethylpropanesulfonic acid (AMPS), etc. Homopolymers based on DADMAC are also obtainable under the trade names Dodigen® 3954, Dodigen 4033 and Genamin® PDAC (Clariant).

Polycondensates which can be used as cationic polymers are obtained by reacting cyanamides with aldehydes and ammonium salts and/or monoamines, by reacting monoamines and/or polyamines with epichlorohydrin, or by reacting polyamines with cyanamides and amidosulfuric acid.

The monoamines used can be primary, secondary and tertiary amines. These may be aliphatic amines, such as, for example, dialkylamines, in particular dimethylamine, alicyclic amines, such as, for example, cyclohexylamine, and aromatic amines, such as, for example, aniline. The amines used can, however, also simultaneously have aliphatic, alicyclic and aromatic substituents. In addition, heterocyclic compounds, such as, for example, pyridine, can also be used.

The term polyamines here includes, for example, diamines, triamines, tetraamines, etc., and the analogous N-alkylpolyamines and the N,N-dialkylpolyamines.

Examples thereof are ethylenediamine, propylenediamine, butylenediamine, pentylenediamine, hexylenediamine, diethylenetriamine, triethylenetetraamine and higher polyamines.

Particularly preferred polyamines are ethylenediamine, diethylenetriamine and dimethylaminopropylamine.

The ammonium salts are salts of ammonia, in particular ammonium chloride or the abovementioned amines or polyamines with various inorganic or organic acids, or else are quaternary ammonium salts.

The cyanamides may be cyanamide or dicyandiamide.

Aldehydes which can be used for the synthesis of the polycondensates are, for example, aliphatic aldehydes, such as, for example, formaldehyde, acetaldehyde, propionaldehyde, butyraldehyde; dialdehydes, such as, for example, glyoxal; unsaturated aldehydes, such as, for example, acrolein, crotonaldehyde and aromatic aldehydes, such as, for example, benzaldehyde. Particular preference is given to the aliphatic aldehydes, in particular formaldehyde.

The cationic polymers can be used in the hair treatment products according to the invention to 0.1 to 5% by weight, preferably to 0.5 to 3% by weight and particularly preferably to 0.5 to 1% by weight.

In a particularly preferred embodiment, the hair treatment products according to the invention comprise, as solvent, propanediol, glycerol or ethanol in concentrations of from 1 to 10% by weight, preferably from 1 to 5% by weight.

In a further particularly preferred embodiment, the pH of the formulations is adjusted to a value between 5 and 12 by adding acidic or alkaline substances. Acidic substances can be, for example, inorganic or organic acids, such as, for example, sulfuric acid, phosphonic acids, citric acid. Alkaline substances are, for example, sodium hydroxide solution, potassium hydroxide solution and soda.

The hair treatment products according to the invention are preferably liquid and clear. However, by adding further ingredients as are customary for such products, it is also possible for opaque or milky cloudy formulations or those with a pearlescent effect to result.

The following further customary constituents can additionally be present in the hair treatment products according to the invention:

anionic, nonionic, cationic, zwitterionic or amphoteric surfactants, and auxiliaries and additives, such as emulsifiers, superfatting agents, biogenic active ingredients, film formers, preservatives, pearlizing agents, dyes.

Anionic surfactants are, in particular, the following compounds and mixtures thereof:

alkali metal salts, ammonium salts, amine salts and salts of amino alcohols of the following compounds: alkylamide sulfates and ether sulfates, alkylaryl polyether sulfates, monoglyceride sulfates, alkylsulfonates, alkylamidesulfonates, alkylarylsulfonates, x-olefinsulfonates, alkyl sulfosuccinates, alkyl ether sulfosuccinates, alkylamide sulfosuccinamates, alkyl sulfoacetates, alkylpolyglycerol carboxylates, alkyl phosphates, alkyl ether phosphates, alkyl sarcosinates, alkyl polypeptidates, alkyl amidopolypeptidates, alkyl ethionates, alkyl taurates, salts of saturated and unsaturated fatty acids, such as oleic acid, ricinoleic acid, palmitic acid, stearic acid, copra oil acid salt or hydrogenated copra oil acid salts, and alkyl polyethoxycarboxylates.

Cationic surfactants are, in particular, long-chain quaternary ammonium compounds, alkylpyridinium salts, fatty amines of polyethers, imidazoline derivatives.

Nonionic surfactants are, in particular, polyethoxylated, polypropoxylated or polyglycerolated ethers of fatty alcohols, polyethoxylated, polypropoxylated and polyglycerolated fatty acid esters, polyethyloxylated esters of fatty acids and of sorbitol, polyethoxylated or polyglycerolated fatty amides.

Further amphoteric or zwitterionic surfactants, additionally to the carbobetaines and sulfobetaines mentioned at the start, are aminoglycinates and amphoteric imidazolinium compounds, cycloimidinium compounds, such as alkylimidazolines, asparagine derivatives, where the alkyl group in these surface-active agents preferably has 1 to 22 carbon atoms.

The total surfactant content of the hair treatment products according to the invention is preferably 5 to 40% by weight, preferably 10 to 30% by weight, in particular 15 to 25% by weight.

The hair treatment products according to the invention can comprise, as auxiliaries and additives, emulsifiers, such as, for example, nonionic emulsifiers, for example oxyethylated or polyglycerolated fatty alcohols, e.g. oleic alcohol, polyoxyethylated with 10 to 30 mol of ethylene oxide, stearyl alcohol with 10 to 15 or 20 mol of ethylene oxide, oleic alcohol, polyglycerolated with 4 mol of glycerol, synthetic fatty alcohols having 9 to 15 carbon atoms, polyoxyethylated with 5 to 10 mol of ethylene oxide, sorbitan esters, monoglycerides, polysorbates, polyethylene glycol mono/di-fatty acid esters, highly ethoxylated fatty acid esters, and high molecular weight silicone compounds, such as, for example, dimethylpolysiloxane and phosphoric acid esters in an amount of from 1 to 25% by weight, or ionic emulsifiers, such as optionally oxyethylated alkyl sulfates, e.g. sodium lauryl sulfate, ammonium lauryl sulfate, ammonium lauryl sulfate, sodium cetylstearyl sulfate, triethanolamine stearyl sulfate, monoethanolamine lauryl sulfate, sodium lauryl ether sulfate, and monoethanolamine lauryl ether sulfate, these latter emulsifiers being present in concentrations between 0.5 and 15% by weight. Superfatting agents which may be used are substances such as, for example, polyethoxylated lanolin derivatives, lecithin derivatives and fatty acid alkanolamides, the latter serving as foam stabilizers at the same time. The thickeners used are preferably hydrogenated castor oil, salts of long-chain fatty acids, preferably in amounts up to 5% by weight and in particular in amounts of from 0.5 to 2% by weight, for example sodium, potassium, aluminum, magnesium and titanium stearates or the sodium and/or potassium salts of behenic acid, and also polysaccharides, in particular xanthan gum, guar guar, agar agar, alginates and tyloses, carboxymethylcellulose and hydroxyethylcellulose, also relatively high molecular weight polyethylene glycol mono- and diesters of fatty acids, polyacrylates, polyvinyl alcohol and polyvinylpyrrolidone, and electrolytes such as sodium chloride and ammonium chloride.

Biogenic active ingredients are to be understood as meaning, for example, plant extracts, protein hydrolyzates and vitamin complexes. Customary film formers are, for example, chitosan, microcrystalline chitosan, quaternized chitosan, polyvinylpyrrolidone, vinylpyrrolidone-vinyl acetate copolymers, polymers of the acrylic acid series, quaternary cellulose derivatives and similar compounds. Suitable preservatives are, for example, phenoxyethanol, formaldehyde solution, parabens, pentadiol or sorbic acid. Suitable pearlizing agents are, for example, glycol distearic esters, such as ethylene glycol distearate, but also fatty acid monoglycol esters or triethylene glycol distearate. Dyes which can be used are the substances approved and suitable for cosmetic purposes, as are listed, for example, in the publication “Kosmetische Fäcrbemittel” [Cosmetic Colorants] from the Dyes Commission of the German Research Society, published in Verlag Chemie, Weinheim, 1984, pp. 81-106. These dyes are usually used in concentrations of from 0.001 to 0.1% by weight, based on the total mixture. The desired viscosity can be established by adding water and/or organic solvents or by adding a combination of organic solvents and thickeners.

With the hair treatment products according to the invention it is possible to improve the shine, the dry combability and the reduction in the regreasing of the hair. Furthermore, an antistatic and feel-improving effect and a pleasant skin feel are found. They have the effect that dry hair is relaxed, shiny and easy to detangle.

The hair shampoos according to the invention are also particularly well suited for colored, tinted or dyed hair since they bring about better fixing of the hair coloration and reduce its washing-out.

### EXAMPLES

### Example 1

A hair shampoo with a total surfactant content of 26% (active ingredient) was prepared.

The anionic surfactants used were alkyl ether sulfate and secondary alkanesulfonate, a betaine, the nonionic surfactant C_{12/14}-alkyl-7EO ethoxylate and the cationic Poly-Dadmac CP 1.

TABLE 1
Anionic hair shampoo comprising CP 7

| | Formulation: |
|---|---|
| Composition: | % active ingredient |
| Alkyl ether sulfate | 5.0% |
| Sec. alkanesulfonate | 1.3% |
| Genagen ® CAB 818 | 3.3% |
| C_{12/14}-alkyl-7EO ethoxylate | 16.3% |
| 1,2-Propanediol | 5% |
| Poly-Dadmac CP 17 | 1% |
| Genapol L-3 | 1% |
| NaCl | 1% |

| | |
|---|---|
| Dye solution | q.s. |
| Preservative | q.s. |
| Water | ad 100% |
| Assessment: | clear solution |

Abbreviations used:
CP = Cationic polymer
CP 1 = polydiallyldimethylammonium chloride, MM = 115.000
Chemical name of the commercial products used:
Genagen CAB 818 cocoamidopropyldimethylcarboxymethyl-ammonium betaine, 30% strength

1. A hair treatment product comprising anionic surfactants, wherein the anionic surfactants are a1) or a2) and b)
a1) alkyl ether sulfate or
a2) alkyl sulfate or a mixture of alkyl ether sulfate and alkyl sulfate.
b) secondary alkanesulfonate,
c1) betaine or
c2) ether carboxylate or a mixture of betaine and ether carboxylate,
d) one or more nonionic surfactants
the mass ratio of the anionic surfactants: the one or more nonionic surfactants is 1:4 to 4:1 and
e) a cationic polymer selected from the group consisting of
homopolymers or copolymers of diallyldimethylammonium chloride,
the reaction products of cyanamides with aldehydes and ammonium salts,
cyanamides with aldehydes and monoamines,
monoamines, polyamines or both with epichlorohydrin, and
polyamines with cyanamides and amidosulfuric acid.

2. The hair treatment product as claimed in claim 1, comprising the components a1 or a2 in concentrations of from 1 to 20% by weight, preferably 2 to 15% by weight, particularly preferably 3 to 12% by weight and very particularly preferably 4 to 10% by weight.

3. The hair treatment product as claimed in claim 1, wherein the mixing ratio of the secondary alkanesulfonate: a1 or a2 is generally 9.9:0.1 parts by weight.

4. The hair treatment product as claimed in claim 1, comprising components c1 or c2 in a concentration of from 0.5 to 15% by weight.

5. The hair treatment product as claimed in claim 1, comprising 1 to 30% by weight, of the one or more nonionic surfactants.

6. The hair treatment product as claimed in claim 1, wherein the one or more nonionic surfactants is an ethoxylate of a synthetic or native alcohol with an HLB value of from 10 to 15.

7. The hair treatment product as claimed in claim 1, wherein the mass ratio of the anionic surfactants: the one or more nonionic surfactants is 1:4 to 4:1.

8. The hair treatment product as claimed in claim 1, wherein the content of both the anionic surfactants and the one or more nonionic surfactants is between 5 and 40% by weight.

9. The hair treatment product as claimed in claim 1, wherein the hair treatment product is clear.

10. The hair treatment product as claimed in claim 1, further comprising at least one solvent selected from the group consisting of propanediol, glycerol, ethanol, n-propanol isopropanol and mixtures thereof, wherein the at least one solvent is present in an amount of from 1 to 10% by weight.

11. The hair treatment product as claimed in claim 1, wherein the hair treatment product is adjusted to a pH between 4.5 and 7.5 and preferably between 5 and 7.

12. A method for exerting a conditioning effect on hair comprising the step of applying a hair treatment product as claimed in claim 1 to the hair.

13. A method for improving the combability of wet and dry hair comprising the step of applying a hair treatment product as claimed in claim 1 to the hair.

14. A method for imparting better shine on hair comprising the step of applying a hair treatment product as claimed in claim 1 to the hair.

15. A method for improving the fixing of hair tints and hair colorations and delays the washing out of hair tints and hair colorations comprising the step of applying a hair treatment product as claimed in claim 1 to the tinted or colored hair.

16. A surfactant system for a hair treatment product comprising a hair treatment product as claimed in claim 1.

17. The hair treatment product as claimed in claim 1, wherein the mass ratio of the anionic surfactants: the one or more nonionic surfactants is 1:2 to 2:1.

18. The hair treatment product as claimed in claim 1, wherein the mass ratio of the anionic surfactants: the one or more nonionic surfactants is 0.8:1 to 1.5:1.

19. The hair treatment product as claimed in claim 1, comprising the components a1 or a2 in concentrations of from 2 to 15% by weight.

20. The hair treatment product as claimed in claim 1, comprising the components a1 or a2 in concentrations of from 3 to 12% by weight.

21. The hair treatment product as claimed in claim 1, comprising the components a1 or a2 in concentrations of from 4 to 10% by weight.

22. The hair treatment product as claimed in claim 1, wherein the mixing ratio of the secondary alkanesulfonate: a1 or a2 is generally 9.8 to 5:0.2 to 5 parts by weight.

23. The hair treatment product as claimed in claim 1, wherein the mixing ratio of the secondary alkanesulfonate: a1 or a2 is generally 4.9 to 2.5:5.1 to 7.5 parts by weight.

24. The hair treatment product as claimed in claim 1, wherein the mixing ratio of the secondary alkanesulfonate: a1 or a2 is generally 2.4 to 0.1:7.6 to 9.9 parts by weight.

25. The hair treatment product as claimed in claim 1, comprising components c1 or c2 in a concentration of from 1 to 10% by weight.

26. The hair treatment product as claimed in claim 1, comprising components c1 or c2 in a concentration of from 2 to 7% by weight.

27. The hair treatment product as claimed in claim 1, comprising 5 to 25% by weight of the one or more nonionic surfactants.

28. The hair treatment product as claimed in claim 1, comprising 10 to 20% by weight of the one or more nonionic surfactants.

29. The hair treatment product as claimed in claim 1, wherein the one or more nonionic surfactants is an ethoxylate of a synthetic or native alcohol with an HLB value of from 11 to 14.

30. The hair treatment product as claimed in claim 1, wherein the mass ratio of the anionic surfactants: the one or more nonionic surfactants is 1:2 to 2:1.

31. The hair treatment product as claimed in claim 1, wherein the mass ratio of the anionic surfactants: the one or more nonionic surfactants is 0.8:1 to 1.5:1.

32. The hair treatment product as claimed in claim 1, wherein the content of both the anionic surfactants and the one or more nonionic surfactants is between 10 to 30% by weight.

33. The hair treatment product as claimed in claim 1, wherein the content of both the anionic surfactants and the one or more nonionic surfactants is between 15 to 25% by weight.

34. The hair treatment product as claimed in claim 10, wherein the at least one solvent is present in an amount of from 1 to 5% by weight.

35. The hair treatment product as claimed in claim 1, wherein the hair treatment product is adjusted to a pH between 5 and 7.

36. A shampoo comprising a hair treatment product as claimed in claim 1.
